(19) United States
(12) Patent Application Publication
(10) Pub. No.: US 2023/0221315 A1
(43) Pub. Date: Jul. 13, 2023

(54) DEVICE OR METHOD FOR DETECTION OF LEUKOCYTE IN DISEASE STATE OR FOR DIAGNOSIS OF LEUKOCYTE-RELATED DISEASE

(71) Applicant: Joo Hun Kang, Ulsan (KR)

(72) Inventors: Joo Hun Kang, Ulsan (KR); Se Yong Kwon, Ulsan (KR); Amanzhol Kurmashev, Ulsan (KR); Brian Choi, Ulsan (KR); Minseok Lee, Ulsan (KR); Sung Jin Park, Ulsan (KR)

(21) Appl. No.: 17/913,974

(22) PCT Filed: Mar. 25, 2021
(86) PCT No.: PCT/KR2021/003695

§ 371 Date: Mar. 15, 2023

(30) Foreign Application Priority Data
Mar. 25, 2020 (KR) 10-2020-0036436

(51) Int. Cl. G01N 33/569

(52) U.S. Cl. CPC G01N 33/56972; G01N 2333/705

(57) ABSTRACT

Provided is a device or method for detection of leukocytes in a disease state or leukocytes in an abnormal state, or diagnosing a leukocyte-related disease, according to the device or method according to an aspect, it is possible to detect leukocytes in a disease state or leukocytes in an abnormal state at an early stage using a small amount of sample isolated from a subject, and thus, there is an effect that allows diagnosis of a leukocyte-related disease, for example, inflammation, an infectious disease, an immune disease, a metabolic disease, or cancer, etc.

### TECHNICAL FIELD

The present disclosure relates to a device or a method for detection of leukocytes in a disease state, or diagnosis of a leukocyte-related disease. This patent application claims priority to Korean Patent Application No. 10-2020-0036436 submitted to the Korean Intellectual Property Office on Mar. 25, 2020, the disclosure of which is incorporated herein by reference.

### BACKGROUND ART

Leukocytes are cells of the immune system that protect the body from infectious diseases and foreign substances, and are cells other than erythrocytes in the blood. When a living body is infected by an external infectious agent, or cancer tissue develops in the body, it is known that leukocytes move and roll along the inner wall of blood vessels, firmly adhere to the inner wall cells of blood vessels, and leak through the vessel walls, by the immune mechanisms of the body.

On the other hand, sepsis is an emergency disease that leads to death within a short period of time by damaging vital organs due to inflammation and an excessive immune response throughout the body caused by infiltration of external infectious agents. Diagnosis of sepsis is made by methods such as culturing infectious bacteria and measuring concentrations of marker proteins in the blood, but the false-negative rate is high and diagnosis takes a long time, even though a prompt treatment is required, making it difficult to make an accurate and effective diagnosis.

In addition, in the case of cancer diseases, the cure rate may be increased when an early diagnosis is made, but the current diagnostic technology for cancer based on gastroscopy, liver ultrasound tests, blood biomarker tests, breast photography, and cervical cell tests requires different diagnosis processes for different types of cancers, and as a result, is expensive, time-consuming, and requires complicated processes, and there is an issue of practical difficulty of an early diagnosis because tests have to be performed frequently for an early diagnosis. As described above, various studies on early diagnosis of leukocytes in a disease state are being conducted (Korean Patent Publication No. 10-1995-7003150), but are still insufficient.

### DESCRIPTION OF EMBODIMENTS

### Technical Problem

An object of the present disclosure is to provide a method or a device for detection of leukocytes in a disease state or diagnosis of cancer or an infectious disease, which is a leukocyte-related disease, even with a small amount of a sample, by using characteristics of leukocytes, in order to solve the above-described issues.

An object of the present disclosure is to provide a method of detecting leukocytes in a disease state including: contacting an isolated biological sample including leukocytes or leukocytes isolated from the biological sample, with leukocyte extravasation factors; and capturing of leukocytes in a disease state by the leukocyte extravasation factors and detecting the captured leukocytes.

Another object of the present disclosure is to provide a method of providing information on diagnosis of a leukocyte-related disease, or diagnosing a leukocyte-related disease, including: contacting an isolated biological sample including leukocytes or leukocytes isolated from the biological sample, with leukocyte extravasation factors; and capturing of leukocytes in a disease state by the leukocyte extravasation factors and detecting the captured leukocytes.

Still another object of the present disclosure is to provide a device for detecting leukocytes in a disease state, including a detector for detecting leukocytes in a disease state including a channel, a particle, a vessel, or a well in which leukocyte extravasation factors are immobilized on a wall of the channel, a surface of the particle, at least a portion of the vessel, or at least a portion of the well, wherein the leukocytes in a disease state in the isolated biological sample are captured by the leukocyte extravasation factors and detected.

Still another object of the present disclosure is to provide a device for diagnosing a disease related to leukocytes, including a detector for detecting leukocytes in a disease state including a channel, a particle, a vessel, or a well, in which leukocyte extravasation factors are immobilized on a wall of the channel, a surface of the particle, at least a portion of the vessel, or at least a portion of the well, wherein leukocytes in a disease state in the isolated biological sample are captured by the leukocyte extravasation factors and detected.

However, the technical problems to be solved by the present disclosure are not limited to the problems mentioned above, and other problems not mentioned will be clearly understood by those of ordinary skill in the art from the following description.

### Solution to Problem

According to an embodiment of the present disclosure, provided is a method of detecting leukocytes in a disease state or leukocytes in an abnormal state, including: contacting an isolated biological sample including leukocytes, or leukocytes isolated from the biological sample with leukocyte extravasation factors; and detecting the captured leukocytes when leukocytes in a disease state or leukocytes in an abnormal state in the sample are captured by the leukocyte extravasation factors.

In addition, according to an embodiment of the present disclosure, provided is a method of providing information on diagnosis of a leukocyte-related disease, or diagnosing a leukocyte-related disease, including: contacting an isolated biological sample including leukocytes or leukocytes isolated from the biological sample with leukocyte extravasation factors; and detecting the captured leukocytes when leukocytes in a disease state or leukocytes in an abnormal state in the sample are captured by the leukocyte extravasation factors.

The term “leukocyte”, used herein, refers to a cell of the immune system that protects the body from infectious diseases and foreign substances, and to a cell other than a erythrocytes in the blood, and specifically, may include granular leukocytes and agranular leukocytes. Examples of granular leukocytes may include neutrophils, basophils, or eosinophils, and examples of agranulocytes may include lymphocytes or monocytes.

In an embodiment, the proportion of neutrophils may be the highest, among the leukocytes captured by the leukocyte extravasation factors. Therefore, it is possible to effectively detect neutrophils in a disease state or neutrophils in an abnormal state by the above method.

The term “leukocyte extravasation”, used herein, may include a series of processes by which the leukocytes are captured by factors of vascular endothelial cells as the leukocytes pass through the blood vessels, roll, adhere, and transmigrate, due to various causes such as tissue damage and infection of the subject, and may refer to a process causing transient, irreversible, and adherent interactions with factors of the vascular endothelial cells. The term “leukocyte extravasation” may be used interchangeably with “leukocyte adhesion cascade”.

The term “leukocyte extravasation factor”, used herein, may refer to a factor involved in leukocyte extravasation as described above. Specifically, leukocyte extravasation factors are expressed in vascular endothelial cells, and may include factors capable of capturing leukocytes along the vascular endothelial cell walls, rolling the leukocytes, and attaching the leukocytes to the vascular endothelial cell wall. The leukocyte extravasation factor may be at least one factor selected from the group consisting of P-selectin, E-selectin, CD34, intercellular adhesion molecule-1 (ICAM-1), soluble ICAM-1, ICAM-2, soluble ICAM-2, glycosylation-dependent cell adhesion molecule-1 (GlyCAM-1), mucosal vascular addressin cell adhesion molecule 1 (MadCAM-1), platelet/endothelial-cell-adhesion molecule (PECAM-1), junctional adhesion molecule A (JAM-A), JAM-B, JAM-C, endothelial cell-selective adhesion molecule (ESAM), vascular cell-adhesion molecule 1 (VCAM-1), cluster of differentiation 99 (CD99), integrins, extracellular domains thereof, cells including the same, membranes of cells including the same, and combinations thereof. Specifically, the leukocyte extravasation factor may be at least one factor selected from the group consisting of ICAM-1, E-selectin, P-selectin, and combinations thereof. According to an embodiment, when the leukocyte extravasation factors are ICAM-1, E-selectin, and P-selectin, detection rates of leukocytes in a disease state or leukocytes in an abnormal state may be significantly increased with the method.

The term “selectin”, used herein, refers to a family of cell adhesion molecules, and includes all of a transmembrane domain of selectin, an N-terminus calcium-dependent lectin domain, an extracellular domain, an EGF-like domain, or a fusion protein of a combination thereof. In addition, the selectin may be L-selectin, P-selectin, E-selectin, or a combination thereof.

The term “integrin”, used herein, may refer to a transmembrane receptor that promotes cell-extracellular matrix adhesion, and may include two subunits of an alpha unit and a beta unit. Specifically, an integrin may include VLA1, VLA2, VLA3, VLA4, VLA5, VLA6, LFA1A, MAC-1, LFA-1, etc., and may include a fusion protein of a combination of subunits.

In an embodiment, the leukocyte extravasation factor may be immobilized on a wall of a channel, a surface of a particle, at least a portion of a vessel, or at least a portion of a well.

The channel may be a microfluidic channel. Specifically, the microfluidic channel may have the leukocyte extravasation factor immobilized on at least a portion of an inner wall surface of the channel.

The width (height, breadth, or diameter) of the cross section of the microfluidic channel may be about 5 μm to about 10,000 μm. Specifically, the width (height, breadth, or diameter) of the cross section of the microfluidic channel may be about 5 μm to about 5,000 μm, about 5 μm to about 2,000 μm, about 5 μm to about 1,000 μm, about 5 μm to about 500 μm, about 5 μm to about 100 μm, about 5 μm to about 50 μm, about 50 μm to about 10,000 μm, about 50 μm to about 5,000 μm, about 50 μm to about 2,000 μm, about 50 μm to about 1,000 μm, about 50 μm to about 500 μm, about 50 μm to about 100 μm, about 100 μm to about 10,000 μm, about 100 μm to about 5,000 μm, about 100 μm to about 2,000 μm, about 100 μm to about 1,000 μm, about 100 μm to about 500 μm, about 500 μm to about 10,000 μm, about 500 μm to about 5,000 μm, about 500 μm to about 2,000 μm, or about 500 μm to about 1,000 μm. According to an embodiment, when the width (height, breadth, or diameter) of the cross-section of the microfluidic channel is less than about 5 μm, considering that the diameter of the leukocytes is 5 μm or more, when an isolated biological sample containing the leukocytes or the leukocytes isolated from the biological sample are injected into the microfluidic channel, movement or flow in the channel is not smooth, and thus, a rate of leukocyte capture by leukocyte extravasation factors, or a detection rate of the captured leukocytes may be significantly reduced, on the other hand, when the width (height, breadth, or diameter) of the cross-section of the microfluidic channel is more than about 10,000 μm, it may be difficult to obtain a meaningful detection result due to difficulties in observation, consumption of a large sample, and low-efficiency reactions between the leukocytes and the extravasation factors.

The term “immobilized”, used herein, may refer to a chemical or a physical bond between a substrate (channel, particle, vessel, or well) and a protein factor (leukocyte extravasation factor). The leukocyte extravasation factor may be immobilized to the channel, particle, vessel, or well by an immobilizing compound or a linker.

The immobilizing compound or linker may refer to a linker for immobilizing leukocyte extravasation factors to a surface of a substrate (channel, particle, vessel, or well). The immobilizing compound or linker may be biotin, avidin, streptavidin, carbohydrate, poly L-lysine, a compound having a thiol group, an amine group, an alcohol group, a carboxyl group, an amino group, a sulfur group, an aldehyde group, a carbonyl group, a succinimide group, a maleimide group, an epoxy group, or an isothiocyanate group, or a combination thereof. Examples of the compound having an amino group include 3-aminopropyltrimethoxysilane, N-(2-aminoethyl)-3-aminopropyltrimethoxysilane (EDA), trimethoxysilylpropyldiethylenetriamine (DETA), 3-(2-aminoethylaminopropyl) trimethoxysilane, 3-aminopropyltriethoxysilane, and the compound having an aldehyde group may include glutaraldehyde. Examples of the compound having a thiol group may include 4-mercaptopropyltrimethoxysilane (MPTS). In addition, examples of the compound having an epoxy group may include 3-glycidoxypropyl trimethoxysilane, and examples of the compound having an isothiocyanate group may include 4-phenylenediisothiocyanate (PDITC), and examples of the compound having succinimide and maleimide groups may include disuccinimidyl carbonate (DSC) or succinimidyl 4-(maleimidephenyl) butyrate (SMPB).

The term “leukocyte in a disease state”, in the present specification, may refer to a state in which movement of a leukocyte is inhibited due to abnormalities in the function of the immune cell, or in the state in which a leukocyte can move to the required site according to the cell signal transduction pathway in order to function as an immune cell, or a state in which expression of related factors is increased or decreased, or in which activation or activity of related factors is inhibited, and may include the meaning of “leukocytes in an abnormal state” or “leukocytes having immune function in an abnormal state”. In the present specification, leukocytes are expressed as in a “disease state” or an “abnormal state” to be distinguished from leukocytes in a “normal state”, however, leukocytes in a disease state or leukocytes in an abnormal state may include leukocytes in a state in which the function and properties as immune cells are in a changed state compared to that of a leukocyte in a normal subject, due to a disease state or an abnormal state of the subject. Accordingly, the meaning of the “leukocyte in a disease state” or “leukocyte in an abnormal state” may include “leukocyte of a subject in a disease state”, “leukocyte isolated from a subject in a disease state”, “leukocytes isolated from a subject in a medically abnormal state”, or “leukocyte isolated from a subject in a medically abnormal state”.

Since the leukocyte may include all kinds of the cells of the immune system in the blood other than erythrocytes (neutrophils, basophils, eosinophils, lymphocytes, monocytes, etc.), the term “neutrophils (or basophils, eosinophils, lymphocytes, monocytes, etc.) in a disease state” or “neutrophils (or basophils, eosinophils, lymphocytes, monocytes, etc.) in an abnormal state”, in the specification, may be defined in the same manner as the description for “leukocyte in a disease state” or “leukocyte in an abnormal state”, or may have the same meaning.

In addition, the leukocyte in a disease state or the leukocyte in an abnormal state or a leukocyte population thereof may have increased or decreased binding capacity with leukocyte extravasation factors compared to the leukocytes in a normal state or a leukocyte population thereof. In an embodiment, the leukocytes in a disease state or the leukocytes in an abnormal state may have increased or decreased expression or activity of factors capable of binding to leukocyte extravasation factors compared to leukocytes in a normal state, or the cell population of the leukocytes in a disease state or the cell population of the leukocytes in an abnormal state may have increased or decreased expression or activity of factors capable of binding to leukocyte extravasation factors compared to a cell population of leukocytes in a normal state.

An increase or decrease in the expression or activity of a factor in the cell population may indicate an increase or decrease in the expression or activity of the factor for a plurality of cells, and not for a single cell, specifically, increase or decrease in the expression or activity of a factor in the cell population may indicate an increase or decrease in the number of cells expressing the factors capable of binding to leukocyte extravasation factors or cells in which the factors capable of binding to leukocyte extravasation factors is activated.

Factors capable of binding to the leukocyte extravasation factors may be at least one selected from the group consisting of sialylated carbohydrates, L-selectin, P-selectin glycoprotein ligand 1 (PSGL-1), and leukocyte function-associated antigen 1 (LFA-1), macrophage-1 antigen (Mac-1; integrin alpha M), VLA-4, CD24, CD44, and E-selectin ligand 1 (ESL-1).

For the isolated biological sample including the leukocytes or the leukocytes isolated from the biological sample in the method, the leukocytes isolated from the biological sample may be a sample including leukocytes isolated from the biological sample. The sample, including the biological sample, may be in a liquid state (for example, blood, suspension, etc.). That is, the isolated biological sample including the leukocytes or the leukocytes isolated from the biological sample (or a sample including the same) may be in a state that leukocytes are included in a liquid (for example, blood, suspension, etc.). Accordingly, the isolated biological sample including the leukocytes or the leukocytes isolated from the biological sample (or a sample including the same) may further include physiological saline, culture solution, etc in addition to the leukocytes.

In an embodiment, in the method, contacting an isolated biological sample including leukocytes or leukocytes isolated from the biological sample, with leukocyte extravasation factors may be performed by injecting the isolated biological sample including the leukocytes or the leukocytes isolated from the biological sample into the channel in which the leukocyte extravasation factors are immobilized on the inner wall. In this regard, the isolated biological sample including the leukocytes or the leukocytes isolated from the biological sample may be injected into the channel in a state in which the leukocytes are included in the liquid (for example, blood, suspension, etc.), and may pass through the channel.

In an embodiment, a concentration of leukocytes included in the isolated biological sample including leukocytes or the leukocytes isolated from the biological sample, which is injected into the channel may be about 10²cells/m1 to about 10⁹ cells/ml, about 10⁴ cells/ml to about 10⁸ cells/ml, about 10⁵ cells/ml to about 10⁷ cells/ml, about 10⁵ cells/ml to about 10⁶ cells/ml, about 10⁶ cells/ml to about 10⁸ cells/ml, or about 10⁶ cells/ml to about 10⁷ cells/ml. According to an embodiment, when the concentration of leukocytes injected into the channel is less than about 10² cells/ml, it may be difficult to obtain a significant detection result, and when the concentration of leukocytes injected into the channel is greater than about 10⁹ cells/ml, it may be difficult to obtain a sample because the required amount of the isolated biological sample including the leukocytes or the leukocytes isolated from the biological sample increases.

In an embodiment, the isolated biological sample including the leukocytes or the leukocytes isolated from the biological sample may be injected into the channel at a flow rate of about 0.1 μL/min to about 100 μL/min, about 1 μL/min to about 50 μL/min, about 2 μL/min to about 40 μL/min, about 3 μL/min to about 30 μL/min, about 4 μL/min to about 20 μL/min, or about 5 μL/min to about 10 μL/min. According to an embodiment, when the flow rate is out of the numerical range, the movement or flow of the isolated biological sample including the leukocytes or the leukocytes isolated from the biological sample in the channel is too slow or too fast, so that detection efficiency, or a leukocyte capture rate of leukocyte extravasation factors, or a detection rate of captured leukocytes may be markedly increased or decreased, and therefore, it may be difficult to obtain a meaningful detection result.

In an embodiment, the detection of the method may be detecting by imaging the captured leukocytes, detecting by fluorescence staining the captured leukocytes, measuring isolated leukocyte lysates by lysing the captured leukocytes, or detecting by attaching a detectable label to the leukocyte or leukocyte extravasation factor.

For the detection, any means suitable for a person skilled in the art that can detect the presence of leukocytes may be used, and the means are not limited to specific means.

For example, the detection may be detection with a camera and/or an image sensor. The camera includes all kinds of cameras and image sensors including digital cameras. In addition, for example, when detecting without fluorescence by using a microscope, leukocytes may be detected by using a device capable of imaging cells by methods such as bright-field, phase-contrast, dark-field, etc. More specifically, leukocytes may be detected by using a camera and an image sensor with or without a magnification lens by a method such as bright-field, phase-contrast, or dark-field. A number of cells may be manually/automatically counted in an image obtained by the imaging by using an image analysis program, or the like.

The fluorescence staining may refer to immunofluorescence staining of cells by using a staining reagent fluorescing green, red, etc., in order that live or dead cells may be visually identified. Through the fluorescence image of the cells obtained by photographing the stained cells, activity, skeleton, etc. of the cells may be identified. For the fluorescence staining, a fluorescent material [calcein AM, fluorescein isothiocuanate (FITC), phalloidin, fluorescein, rhodamine, 6-carboxy-tetramethyl-rhodamine (TAMRA), Cy-3, Cy-5, Texas Red, 4,6-diamidino-2-phenylindole (DAPI), Hoechst staining, Dil Stain (DilC₁₈(3)), and coumarin], fluorescent dyes (Alexa Fluor 610, Alexa Fluor 647 (Life Technology), DyLight 633, DyLight 650, DyLight 680 (Thermo Fisher), TF5, TF6, TF7 (ACZO Biotech), Quantum dots, etc.) and particles containing fluorescent dyes (Flash Red (Bangs Labs), Dark Red, Infrared (Invitrogen), Sky Blue (Sperotech), etc.) may be used, but the fluorescence staining is not limited thereto.

For the detection of the method, measuring the isolated leukocyte lysate by lysing the captured leukocytes may be, specifically, isolating the leukocyte lysate by destroying the leukocytes captured by the leukocyte extravasation factor, by using a leukocyte lysing agent (for example, a surfactant such as SDS), and measuring the isolated leukocyte lysate, but is not limited thereto. The isolated leukocyte lysate may be a nucleic acid, a cell membrane protein, a cytoplasmic protein, or a nuclear protein, but is not limited thereto. Polymerase chain reaction (PCR), fluorescence in situ hybridization (FISH), enzyme-linked immunosorbent assay (ELISA), Western blot, or immune-detection (for example, immunodetection method using an antibody, etc.) may be used to measure the isolated leukocyte lysate, but the method is not limited thereto.

The detectable label may be an optical label, an electrical label, a magnetic label, or an indirect label. The optical label is a material that generates a detectable optical signal, and may be a radioactive material, or a chromogenic material such as a fluorescent material. The indirect label refers to a substance capable of generating a detectable label as a result of binding to a specific substance, such as an enzyme that converts a substrate into a chromogenic substance, or a substrate of the enzyme, an antibody, or an antigen.

In an embodiment, the method may include counting a total number of leukocytes per unit sample volume in an isolated biological sample including the leukocytes, or isolating the leukocytes from the isolated biological sample including the leukocytes and counting the same. For example, counting a total number of leukocytes may be counting a total number of leukocytes per unit sample volume included in the isolated biological sample including the leukocytes or the leukocytes isolated from the biological sample, in a state in which the isolated biological sample including the leukocytes or the leukocytes isolated from the biological sample are not contacted with the leukocyte extravasation factors.

The counting may be performed before, after, or during the detection of the leukocytes.

In an embodiment, the method may further include isolating leukocytes in the isolated biological sample including the leukocytes. The isolating may further include counting the number of isolated leukocytes. The process of isolating leukocytes in the isolated biological sample may include, for example, injecting the isolated biological sample into a channel including a microstructure array, so that leukocytes having a predetermined size may be temporarily captured in the microstructure array, or when the isolated biological sample is whole blood, selectively hemolyzing erythrocytes from the whole blood and then centrifuging to separate leukocytes, but is not limited thereto. The isolating may be performed during or before the detection of the leukocytes.

In an embodiment, the detecting in the method may include analyzing a ratio of the number of leukocytes captured by the extravasation factors to the total number of counted leukocytes per unit sample volume, or analyzing the number of leukocytes captured by the extravasation factors among the number of the isolated leukocytes.

In an embodiment, the detecting in the method may further include: analyzing a ratio of the number of leukocytes captured by the extravasation factors as leukocytes isolated from the sample of the subject for the test to the total number of leukocytes per unit sample volume included in the sample of the subject for the test; and/or comparing the analyzed ratio with a ratio of the number of leukocytes captured by the extravasation factors as leukocytes isolated from the sample of a normal subject to the total number of leukocytes per unit sample volume included in the sample of the normal subject; and/or when the ratio analyzed from the sample of the subject for the test is higher or lower than the ratio analyzed from the sample of a normal subject, determining the leukocytes to be leukocytes in a disease state, or leukocytes in an abnormal state, or determining the subject to be in a disease state, or in a medically abnormal state.

In an embodiment, in the case of collecting and using an arbitrary number of previously isolated leukocytes, the detecting may further include: analyzing the number of leukocytes captured by the extravasation factors among the isolated and counted leukocytes; and/or determining the leukocytes as leukocytes in a disease state or leukocytes in an abnormal state, or determining the subject to be in a disease state or a medically abnormal state, when the analyzed number of the leukocytes is higher or lower compared to the number of the normal leukocytes captured by the extravasation factors (or leukocytes isolated from a sample of a normal subject).

In addition, the detecting may include determining a subject to be in a disease state, or a medically abnormal state, when the number of detected leukocytes (for example, an optical signal, etc.) increases or decreases compared to leukocytes in a normal state or leukocytes isolated from a sample of a normal subject (for example, a reference signal).

The ratio of the number of leukocytes captured by the extravasation factors as leukocytes isolated from the sample of a normal subject to the total number of leukocytes per unit sample volume included in the sample of a normal subject may be additionally determined in a normal subject, or pre-determined as a reference value.

In an embodiment, the disease state or the leukocyte-related disease may include inflammation, an infectious disease, an immune disease, a metabolic disease, cancer, or cancer metastasis.

The meaning of the term “infectious disease”, used herein, may include systemic or local infections of viruses, bacteria, mold, or fungi, or sepsis, bacteremia, or viremia. For example, the bacterial or fungal infections may include diseases and symptoms resulting from infection of Pseudomonas, Escherichia, Klebsiella, Enterobacter, Proteus, Serratia, Candida, Staphylococci, Streptococci, Chlamydia, Mycoplasma, and various other species. For example, the viral infections may include diseases and symptoms resulting from infection of influenza virus, coronavirus, adenovirus, parainfluenza virus, rhinovirus, respiratory syncytial virus (RSVs), herpes virus, cytomegalovirus, hepatitis virus such as hepatitis B and C, and various other species.

The term “immune disease”, used herein, refers to any disease that stimulates the immune system (i.e., causes an immune activation state or an immune inactivation state), or any disease caused by immune stimulation (immune activation), immune hyperactivity, immune inactivation, or immunosuppression, for example, may be at least one selected from the group consisting of systemic or local infection of viruses, bacteria, mold, or fungi (for example, initial infection, long-term infection, etc.), inflammation (for example, acute inflammation or chronic inflammation), sepsis, bacteremia, cancer, cancer metastasis, autoimmune diseases, and cardiovascular diseases (arteriosclerosis, stroke, etc.). More specifically, the immunity-related disease may include a disease related to, or caused by the state of immune stimulation (immune activation), or the state of abnormal immunity (i.e., immune hyperactivity), such as systemic or local infection, acute inflammation, sepsis, bacteremia, an autoimmune disease, a cardiovascular disease (arteriosclerosis, stroke, etc.) as described above; or a disease related to, or caused by the state of abnormal immunity (i.e., immune inactivation or reduced immunity), such as long-term infection, chronic inflammation, cancer, cancer metastasis, and the like.

The term “inflammation”, used herein, is a result of a localized protective response of body tissues against host invasion, usually due to foreign substances or harmful stimuli, and causes of this inflammation may be infectious agents such as bacteria, viruses, and parasites, physical causes such as burns or radiation, or chemicals such as toxins, drugs or industrial reagents, or immune responses such as allergies, and autoimmune reactions, or an abnormal state related to oxidative stress. Examples of the inflammatory disease of the present disclosure are acute bronchitis, chronic bronchitis, acute bronchiolitis, chronic bronchiolitis, sepsis, septic shock, acute respiratory distress syndrome, multiple organ failure, or chronic obstructive pulmonary disease.

The term “metabolic disease”, used herein, may refer to a disease caused by an imbalance in the chemical composition of the body, such as hormones, carbohydrates, lipids, proteins, vitamins, minerals, or water. Examples of metabolic diseases of the present specification may include obesity, diabetes (for example, type I or type II diabetes), insulin resistance, atherosclerosis, arteriosclerosis, hepatic steatosis, fatty liver, hypertension, hypercholesterolemia, dyslipidemia, or hyperlipidemia.

The term “cancer”, used herein, refers to a group of diseases characterized by excessive cell proliferation and infiltration into surrounding tissues when a normal balance of apoptosis is disrupted. The cancer may be selected from the group consisting of carcinoma derived from epithelial cells, such as lung cancer, laryngeal cancer, stomach cancer, colon/rectal cancer, liver cancer, gallbladder cancer, pancreatic cancer, breast cancer, cervical cancer, prostate cancer, kidney cancer, skin cancer, etc., sarcoma derived from connective tissue cells, such as bone cancer, muscle cancer, adipose cancer, and fibroblast cancer, hematological cancers derived from hematopoietic cells, such as leukemia, lymphoma, and multiple myeloma, and tumors occurring in nerve tissues.

The term “isolated biological sample”, used herein, may refer to a biological sample isolated from a subject.

In an embodiment, the isolated biological sample may include samples such as, tissues, cells, whole blood, blood, serum, plasma, lymphatic fluid, bone marrow, tissue fluid, synovial fluid, saliva, nasal fluid, sputum, cerebrospinal fluid, ocular fluid, and urine isolated from the body of a subject. The biological sample may be isolated from a subject suspected of having a disease state or a medically abnormal state.

In an embodiment, a subject refers to any animal, including humans, and more specifically, includes mammals such as humans, or non-human primates, mice, dogs, cats, horses, and cows. In addition, the subject may be a subject suspected to be in a disease state or a medically abnormal state, or a subject determined to have a disease state or a medically abnormal state.

Without being limited by any particular theory, as schematized in FIG. 1, leukocytes in a disease state or leukocytes in an abnormal state according to an embodiment (or leukocytes from a diseased subject or a subject in a medically abnormal state) has increased expression or activation, or decreased expression or inhibited activation (on the surface of the leukocyte) of factors related to leukocyte extravasation (for example, factors capable of binding to leukocyte extravasation factors). Accordingly, as FIG. 2 is an example of a case in which factors capable of binding to the leukocyte extravasation factors are activated, or expression of the factors is increased, as schematized in FIG. 2, when expression of the factors related to leukocyte extravasation increases, or the number of leukocytes with increased expression increases, the leukocytes in a disease state or leukocytes in an abnormal state (or leukocytes from a diseased subject or a subject in a medically abnormal state) becomes a state capable of binding more, or binding more strongly to leukocyte extravasation factors, compared with leukocytes in a normal state (or leukocytes of a normal subject), or a number of the leukocytes in such a state increases, and using these characteristics of leukocytes, by contacting leukocyte extravasation factors immobilized or attached to a channel, a particle, a container, or a well with leukocytes isolated from a subject, leukocytes in a disease state or leukocytes in an abnormal state (or leukocytes from a diseased subject or a subject in a medically abnormal state) may be detected. For example, when leukocytes in a disease state or leukocytes in an abnormal state (or leukocytes from a diseased subject or a subject in a medically abnormal state) are contacted with the leukocyte extravasation factors, more numbers of leukocytes may be captured by the leukocyte extravasation factors and be detected, compared to when leukocytes in a normal state (or leukocytes of a normal subject) are brought into contact with the leukocyte extravasation factors. On the other hand, when expression of factors related to leukocyte extravasation factors is decreased, or when leukocytes having decreased expression increases in the leukocytes in a disease state or leukocytes in an abnormal state (or leukocytes from a diseased subject or a subject in a medically abnormal state), the leukocytes in a disease state or leukocytes in an abnormal state (or leukocytes from a diseased subject or a subject in a medically abnormal state) becomes a state capable of binding less, or binding more weakly with the leukocyte extravasation factors, compared with the leukocytes in a normal state (or leukocytes of a normal subject), or a number of leukocytes in such a state increases, and using these characteristics of leukocytes, by contacting leukocyte extravasation factors immobilized or attached to a channel, a particle, a container or a well with leukocytes isolated from a subject, leukocytes in a disease state or leukocytes in an abnormal state (or leukocytes from a diseased subject or a subject in a medically abnormal state) may be detected. For example, when leukocytes in a disease state or leukocytes in an abnormal state (or leukocytes from a diseased subject or a subject in a medically abnormal state) are contacted with the leukocyte extravasation factors, fewer numbers of leukocytes may be captured by the leukocyte extravasation factors and be detected, compared to when leukocytes in a normal state (or leukocytes of a normal subject) are brought into contact with the leukocyte extravasation factors.

The method of providing information on diagnosis of a leukocyte-related disease, or diagnosing a leukocyte-related disease may include a method of providing information on the progress of the disease or state of disease progression (for example, progression of cancer or inflammation, cancer stage, cancer metastasis, etc.) of a subject which has already been determined to have the leukocyte-related disease.

Therefore, the method of providing information on diagnosis of a leukocyte-related disease, or diagnosing a leukocyte-related disease may include a method of providing information on the progress of the leukocyte-related disease or the state of the disease progression, including: contacting the biological sample isolated from the subject including the leukocytes of the subject already determined to have a leukocyte-related disease, or the leukocytes isolated from the biological sample with the leukocyte extravasation factors; and detecting the captured leukocytes when the leukocytes in a disease state or leukocytes in an abnormal state are captured by the leukocyte extravasation factors.

In the method of providing information on the progress of the leukocyte-related disease or the state of the disease progression, the process of detecting may further include: quantitatively evaluating the leukocytes captured by the leukocyte extravasation factors and detected; and/or comparing the numerical value obtained in the process of quantitatively evaluating with one or more reference values; and/or when the value obtained in the process of quantitatively evaluating is the same as a specific reference value among the one or more reference values, or is within a range of a specific reference value, or is higher or lower than a specific reference value, determining the progress of the disease or the state of the disease progression of the subject determined to have a leukocyte-related disease (for example, progression of cancer or inflammation, cancer stage, cancer metastasis, etc.).

The reference value may be a numerical range. In addition, the reference value may be predetermined and may be a value standardized by a statistical analysis. For example, the reference value may be predetermined, and may be a value obtained by quantitatively evaluating the leukocytes detected by contacting leukocytes isolated from one or more other subjects determined to have the same disease as the subject of the test, that is, the subject already determined to have a leukocyte-related disease, with the leukocyte extravasation factors. In this regard, the reference value may be a plurality of numerical values or numerical ranges obtained by repeatedly evaluating according to the progress of the disease or the state of the disease progression.

According to an embodiment of the present disclosure, provided is a device for detecting leukocytes in a disease state or leukocytes in an abnormal state, including a detector of leukocytes in a disease state or leukocytes in an abnormal state, including a channel, a particle, a vessel, or a well, in which leukocyte extravasation factors are immobilized on a wall of the channel, a surface of the particle, at least a portion of the vessel, or at least a portion of the well, wherein the leukocytes in a disease state or the leukocytes in an abnormal state in the isolated biological sample are captured by the leukocyte extravasation factors and detected.

In addition, according to an embodiment of the present disclosure, provided is a device for diagnosing a leukocyte-related disease, including a detector of leukocytes in a disease state or leukocytes in an abnormal state, including a channel, a particle, a vessel, or a well, in which leukocyte extravasation factors are immobilized on a wall of the channel, a surface of the particle, at least a portion of the vessel, or at least a portion of the well, wherein the isolated leukocytes in a disease state or leukocytes in an abnormal state in the biological sample are captured by the leukocyte extravasation factors and detected.

The channel may be a microfluidic channel.

The device may further include an inlet, into which the isolated biological sample is injected, and/or an outlet through which the analyzed sample is discharged.

In an embodiment, the device may further include a separator for separating leukocytes from the isolated biological sample. The leukocytes separated by the separator may be injected into the detector. The process or method of isolating leukocytes from the isolated biological sample that may be performed in the separator is as described above. In addition, the device may further include an analysor including a means of detection capable of detecting captured leukocytes.

The device may be a diagnostic kit, a detection kit, a microfluidic device, or a microfluidic chip.

Any of the terms or elements mentioned in the description of the device which is the same as mentioned in the description of the method is understood to be the same as mentioned in the description of the method above.

### Advantageous Effects of Disclosure

According to a device or a method for detection of leukocytes in a disease state or leukocytes in an abnormal state, or diagnosing a leukocyte-related disease, according to an aspect, it is possible to detect leukocytes in a disease state or leukocytes in an abnormal state at an early stage using a small amount of sample isolated from a subject, and thus, there is an effect that allows diagnosis of a leukocyte-related disease, for example, inflammation, an infectious disease, an immune disease, a metabolic disease, or cancer, etc.

### BRIEF DESCRIPTION OF DRAWINGS

FIG. 1 shows a diagram schematically illustrating a difference between a surface receptor of a leukocyte in a normal state and a surface receptor of a leukocyte in a disease state, according to an embodiment.

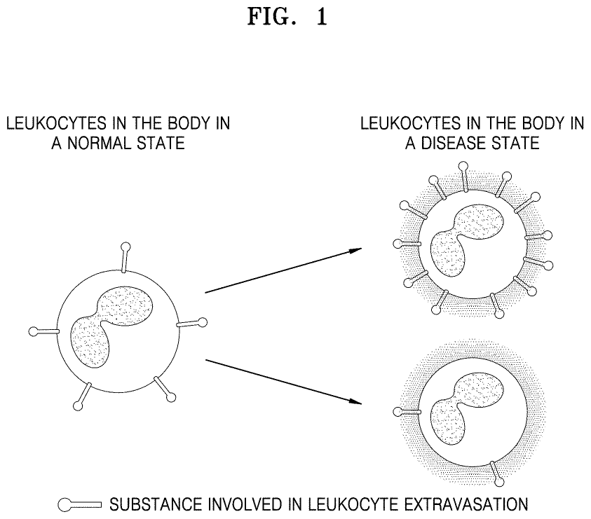

FIG. 2 shows a diagram schematically showing a principle of a method and a device for detecting leukocytes in a disease state, according to an embodiment.

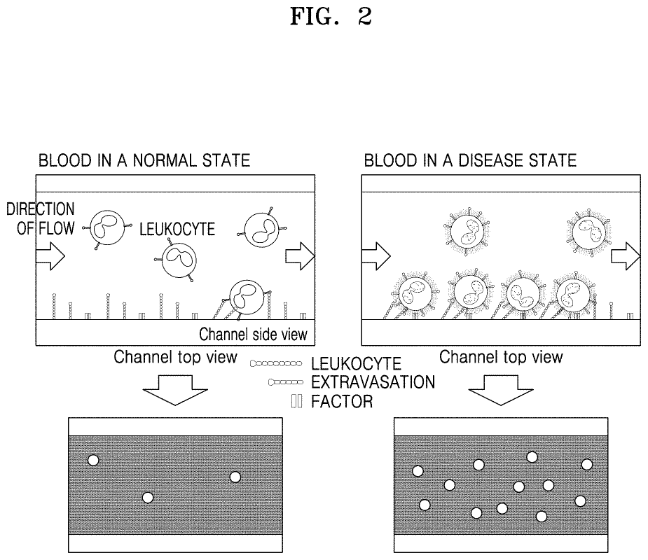

FIG. 3 shows a graph showing degrees of adhesion of leukocytes in a normal state and leukocytes in a sepsis state to a channel, by using a leukocyte extravasation factor, according to an embodiment.

FIG. 4 shows an image showing the results of fluorescence staining of leukocytes in a normal state or a sepsis state, attached to a channel by using leukocyte extravasation factors, according to an embodiment.

FIG. 5 shows a graph showing average expression levels of PSGL-1 protein of leukocytes in a normal state or a sepsis state, according to an embodiment.

FIG. 6 shows a graph showing (number of leukocytes attached to the channel)/(total number of leukocytes in the sample) % of leukocytes in a normal state or a sepsis state, according to an embodiment.

FIG. 7 shows results showing (number of leukocytes attached to the channel)/(total number of leukocytes in the sample) % of leukocytes in a normal state or a sepsis state, according to an embodiment.

FIG. 8 shows an image showing results of fluorescence staining of leukocytes in a normal state or a cancer state, which are attached to a channel by using leukocyte extravasation factors, according to an embodiment.

FIG. 9 shows results showing (number of leukocytes attached to the channel)/(total number of leukocytes in the sample) % of leukocytes in a normal state or a cancer state, according to an embodiment.

FIG. 10A shows results showing the time-dependent change of tumor size after inoculation of cancer cells in a mouse tumor model.

FIG. 10B shows results showing the time-dependent change of tumor weight after inoculation of cancer cells in a mouse tumor model.

FIG.11 shows results of counting leukocytes in a normal state or a cancer state, which are attached to a channel by using leukocyte extravasation factors, according to an embodiment.

FIG. 12 shows results showing the proportion of neutrophils among leukocytes attached to a channel by using leukocyte extravasation factors, according to an embodiment.

FIG. 13A shows a photograph of leukocytes attached to a channel by using leukocyte extravasation factors, imaged with a fluorescence microscope, according to an embodiment.

FIG. 13B shows a photograph of leukocytes attached to the same channel as in FIG. 13A, imaged under a microscope on a Bright field (BF).

FIG. 13C is a photograph (ImageJ, USA), in which the number of cells was automatically counted by using the image taken under a microscope on the BF of FIG. 13B.

FIG. 13D shows a graph showing the difference between counting leukocytes attached to a channel by using leukocyte extravasation factors in a fluorescence image (CT) and a Bright field (BF) image, according to an embodiment.

### MODE OF DISCLOSURE

Hereinafter, the present disclosure will be described in more detail through examples. However, these examples are intended to illustrate the present disclosure, and the scope of the present disclosure is not limited to these examples.

### EXAMPLE 1

### Preparation of Microfluidic Chip Including Microfluidic Channel for Detecting Leukocytes in a Disease State

In order to detect leukocytes in a disease state, a microfluidic chip including a microfluidic channel coated with leukocyte extravasation factors was prepared.

Specifically, polydimethylsiloxane (PDMS) including a surface having a pattern of a channel (width: about 400 μm, height: about 100 μm, length: about 27 mm) was prepared. The surface of the PDMS having the pattern of a channel was treated with air plasma and treated with about 10% 3-aminopropyltriethoxysilane (APTES) dissolved in about 99.9% ethanol. Thereafter, the surface of PDMS treated with APTES was bonded to a glass slide (LumiNano, Korea), in which aldehyde groups were activated, and reacted at about 37° C. for about 5 hours to prepare a microfluidic chip including microfluidic channels.

Then, the inside of the microfluidic channel was coated with leukocyte extravasation factors. Specifically, ICAM-1+E-selectin; and 1×PBS (pH 7.4) including ICAM-1+E-selectin+P-selectin (each about 5 μg/ml) were injected through the inlet to the microfluidic channel at a rate of about 10 μL/min for about 2 to 4 minutes. In this state, after stopping the operation of the micropump controlling the flow of the fluid, the liquid injected into the microfluidic channel was left for about 30 minutes at room temperature to induce the proteins to attach to the aldehyde group. After that, 1×PBS (pH 7.4) including about 3% bovine serum albumin was injected into the channel at a flow rate of about 10 μL/min for about 4 minutes, and after stopping the micropump, the liquid was left in the channel for about 1 hour, to undergo a blocking process to prevent non-specific reactions, and 1×PBS (pH 7.4) was injected into the channel at a flow rate of about 10 μL/min for about 4 minutes to wash.

As a result, a microfluidic chip including a microfluidic channel coated with leukocyte extravasation factors was obtained. Treatment of all samples were processed through the inlet and outlet provided in the microfluidic chip.

### EXPERIMENTAL EXAMPLE 1

### Detection of Leukocytes in Sepsis State Using Leukocyte Extravasation Factors

In order to detect leukocytes in a sepsis state by using leukocyte extravasation factors, the following experiment was performed.

First, sepsis was induced by intraperitoneally injecting E. coli K12 (about 10⁸ CFU/1 mL physiological saline) into 9-week-old Wistar male rats.

Then, about 50 μL of the blood of prepared sepsis-induced rat or normal rat was collected, mixed with ACK lysis buffer at a ratio of about 1:20, and reacted at room temperature for about 5 minutes, and then centrifuged to isolate the leukocytes. The isolated leukocytes were washed with 1×PBS, and then diluted in about 100 μL of 1×PBS to prepare a biological sample including leukocytes. In the same manner as in Example 1, a microfluidic chip including a microfluidic channel in which rat ICAM-1, rat P-selectin, and rat E-selectin, etc. were immobilized was prepared (each using a solution at a concentration of about 5 μg/ml), and after injecting and flowing the biological sample into the inlet of the prepared channel for about 10 minutes at a rate of about 8 μl/min, the channel was washed for about 4 minutes by injecting 1×PBS into the channel at a flow rate of about 8 μl/min, and this was repeated about two more times, in order to remove the leukocytes not attached to the channel. Then, in order to stain the leukocytes attached or trapped in the microfluidic channel, the inside of the channel was filled with dyes such as Hoechst and Cell tracker, and incubated at room temperature for about 20 minutes, and the channel was washed for about 4 minutes by injecting 1×PBS into the channel at a flow rate of about 8 μl/min. After that, the inside of each channel was photographed with a fluorescence microscope, and images inside each channel to which leukocytes were attached were captured, and the total number of leukocytes captured in the channel was counted.

About 50 μL of blood was separately collected from sepsis-induced or normal rats prepared for total leukocyte count analysis of the sample, and leukocytes were isolated in the same manner as described above, and the isolated leukocytes were diluted in about 100 μL of 1×PBS to prepare a biological sample, and the total number of leukocytes was counted by using a hemocytometer after staining with CellTracker, DAPI, or Hoechst.

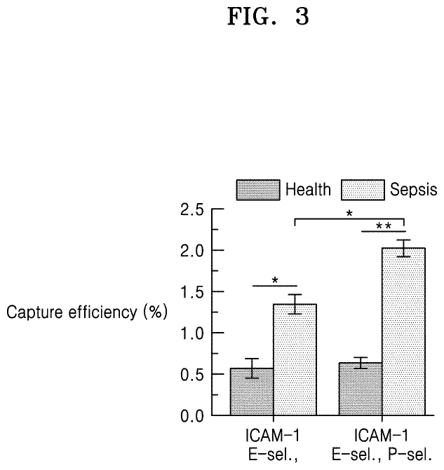

Thereafter, ratios of leukocytes captured in each channel of the control group (normal rat) and the sepsis group were compared using (leukocytes attached to the channel)/(number of total leukocytes in the sample) (%), and the results are shown in FIG. 3.

As shown in FIG. 3, as a result of immobilizing leukocyte extravasation factors according to an embodiment to each channel, and capturing leukocytes in a sepsis state (leukocytes of a sepsis rat) and leukocytes in a normal state (leukocytes of a normal rat) in each channel, more leukocytes in a sepsis state were attached to the channel than the leukocytes in a normal state.

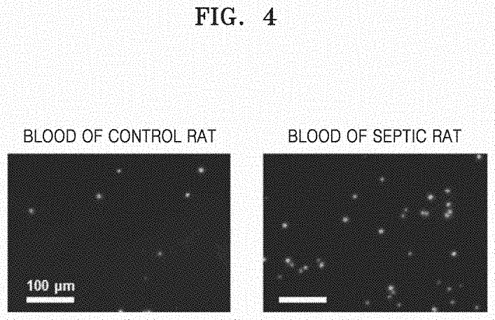

In addition, the cells stained with CellTracker in a channel in which ICAM-1+E-selectin+P-selectin, etc were immobilized were photographed with a fluorescence microscope, and the results are shown in FIG. 4.

As shown in FIG. 4, as a result of staining the leukocytes attached to each channel with CellTracker, more leukocytes in a sepsis state were attached to the channels than leukocytes in the normal control group.

These results indicate that, leukocytes in a disease state or leukocytes in an abnormal state have increased expression of factors related to leukocyte extravasation (for example, factors capable of binding to leukocyte extravasation factors), or in the subject having a disease, the number of leukocytes with an increased expression level of the factor increases. And these results indicate that, when the expression of the factors related to leukocyte extravasation are increased, the leukocytes in a disease state or leukocytes in an abnormal state become a state capable of binding more strongly to the leukocyte extravasation factors compared with normal leukocytes, and using such characteristics of leukocytes, by contacting leukocyte extravasation factors immobilized on the channel, particle, vessel, or well, with the leukocytes isolated form the subject, leukocytes in a disease state or leukocytes in an abnormal state may be detected.

### EXPERIMENTAL EXAMPLE 2

### Identification of Expression Factors of Leukocytes in Disease State

It was confirmed whether the expression of proteins interacting with leukocyte extravasation factors was actually increased in leukocytes in a disease state.

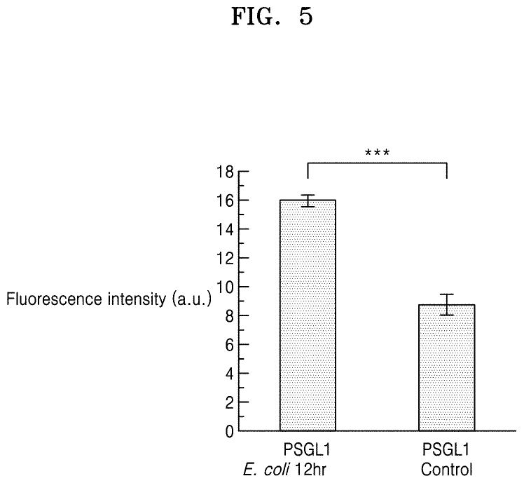

Specifically, in the same manner as in Experimental Example 1, leukocytes were isolated from sepsis-induced rats and normal rats, and the leukocytes were fluorescence stained. Average expression levels of PSGL-1 of the isolated leukocytes were compared by measuring the fluorescence intensity at the single cell level, and the results are shown in FIG. 5.

As shown in FIG. 5, it was confirmed that the expression of PSGL-1 in leukocytes of sepsis-induced rats was increased by about two times compared to leukocytes of normal rats.

These results indicate that, expression of factors interacting with leukocyte extravasation factors increases in leukocytes in a disease state or leukocytes in an abnormal state, and by using the leukocyte extravasation factors according to an embodiment, it is possible to diagnose a disease that increases expression of factors related to the surface of a leukocyte, such as sepsis.

### EXPERIMENTAL EXAMPLE 3

### Early Detection of Sepsis by Using Leukocyte Extravasation Factors

It was confirmed whether sepsis may be diagnosed early by using leukocyte extravasation factors.

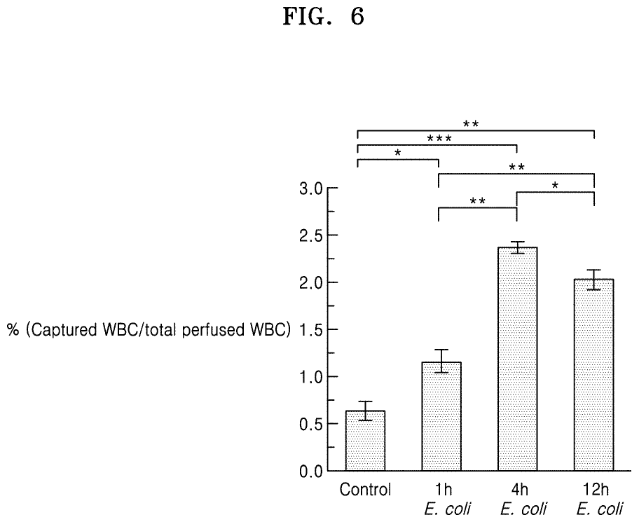

Specifically, in the same manner as in Experimental Example 1, (number of leukocytes attached to the channel)/(total number of leukocytes in the sample) % was measured over time after bacterial inoculation, and the results are shown in FIG. 6.

As shown in FIG. 6, it was found that the numerical values of the sepsis group and the control group were significantly different even after about 1 hour after bacterial inoculation.

These results indicate that sepsis may be detected early even with a very small sample by using the leukocyte extravasation factors according to an embodiment.

### EXPERIMENTAL EXAMPLE 4

### Diagnosis of Infectious Disease or Inflammation Induced by Various Infectious Agents Using Leukocyte Extravasation Factors

It was confirmed whether an infectious disease or inflammation caused by various infectious agents may be diagnosed by using leukocyte extravasation factors.

First, 1 mL of physiological saline (sham control), E. coli K12 (about 10⁸ CFU/1 mL physiological saline), lipopolysaccharide (LPS; about 5 mg/kg), and methicillin-resistant Staphylococcus aureus (MRSA; about 10⁸ CFU/1 mL physiological saline) were respectively intraperitoneally injected into 8-week-old male Wistar rats, and the rats were bred for about 4 hours to prepare rats with infectious diseases or inflammation induced by various sepsis-inducing substances and bacteria.

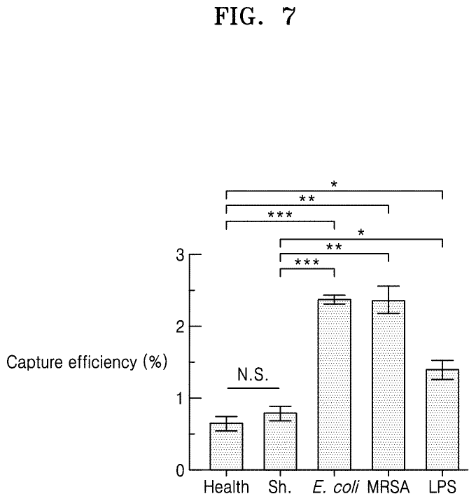

In the same manner as in Example 1, a microfluidic chip including a microfluidic channel in which rat ICAM-1, rat P-selectin, and rat E-selectin, etc. were immobilized was prepared (each using a solution at a concentration of about 5 μg/ml), and in the same manner as in Experimental Example 1, after injecting the leukocytes isolated from the rat into the microfluidic channel, (number of leukocytes attached to the channel)/(total number of leukocytes in the sample) % was measured, and the results are shown in FIG. 7.

As shown in FIG. 7, as a result of using the leukocytes of the sham control group, and the leukocytes in a sepsis state caused by various causes, and the leukocytes in an inflammatory state, in the channel injected with leukocytes of the sham control group (rats in which only physiological saline was injected intraperitoneally) and the control group (normal rats), there was no difference in the percentage of leukocytes attached to each channel, but it was found that the number of leukocytes attached to the channel was remarkably increased in the channel into which the leukocytes of the experimental group with infectious diseases or inflammation induced by various infectious agents such as E. coli, MRSA, and LPS were injected.

These results indicate that by detecting leukocytes in a state with various infectious diseases or inflammation, by using leukocyte extravasation factors according to an embodiment, it is possible to diagnose a corresponding disease.

### EXPERIMENTAL EXAMPLE 5

### Early Detection of Cancer by Using Leukocyte Extravasation Factors

The following experiment was conducted to confirm whether cancer may be diagnosed early by using leukocyte extravasation factors.

First, 4T1 cancer cells (breast cancer cells) were cultured in RPMI 1640 medium containing about 10% fetal bovine serum (FBS) and about 1% antibiotics in an incubator at about 37° C. and under the condition of 5% CO₂. The medium was changed about once every 2 days to 3 days, and the cells were subcultured using a trypsin/EDTA solution of about 0.25% when the flask was about 80% full with the cells.

Then, the cultured 4T1 cancer cells were injected into the mammary fat pad of 8-week-old female BALB/C mice at a concentration of about 3×10⁶ cells/0.1 mL 1×PBS (pH 7.4). After injection, they were bred in cages for one week.

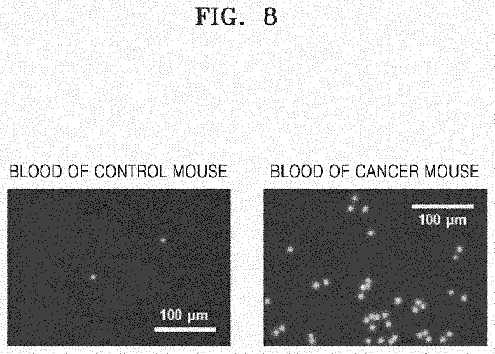

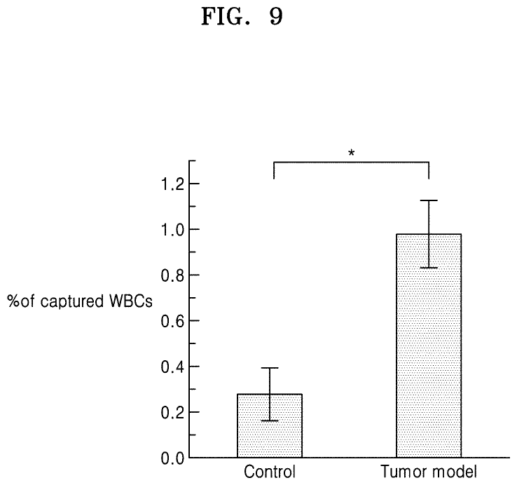

In the same manner as in Example 1, a microfluidic chip including a microfluidic channel in which mouse ICAM-1, mouse P-selectin, and mouse E-selectin, etc. were immobilized was prepared (each using a solution at a concentration of about 5 μg/ml), and about 50 μL of the blood of the prepared cancer-induced mice or non-cancerous mice was collected, mixed with ACK lysis buffer in a ratio of about 1:20, and reacted at room temperature for about 5 minutes, and then the leukocytes were isolated by using centrifugation. The isolated leukocytes were washed with 1×PBS, and then diluted in about 100 μL of 1×PBS to prepare a biological sample including leukocytes. After injecting a sample including leukocytes isolated from the mice into the microfluidic channel, the cells captured in the channel were photographed with fluorescence staining, and (number of leukocytes attached to the channel)/(number of total leukocytes in the sample) % was measured, and the results are shown in FIGS. 8 and 9, respectively.

As shown in FIGS. 8 and 9, when leukocytes of cancer-induced mice were injected into the channel, it was found that significantly more leukocytes were attached to the channel and detected than when leukocytes of a control mouse were injected into the channel, and it was found that cancer may be diagnosed at an early stage in a subject with cancer, even when it was only about a week after the onset of cancer.

These results indicate that in leukocytes in a disease state or in leukocytes in an abnormal state, expression of factors that interact with leukocyte extravasation factors increases or a number of leukocytes with increased expression of factors that interact with leukocyte extravasation factors increases, and it is possible to diagnose a disease that increases expression of surface-related factors in a leukocyte, such as cancer, by using leukocyte extravasation factors according to an embodiment. In particular, these results mean that cancer may be diagnosed at an early stage with a very small sample using the leukocyte extravasation factor according to an embodiment.

### EXPERIMENTAL EXAMPLE 6

### Detection of Cancer by Using Leukocyte Extravasation Factors

In order to confirm whether cancer may be diagnosed using leukocyte extravasation factors, the following experiment was conducted.

First, 4T1 cancer cells were cultured in the same manner as in Experimental Example 5.

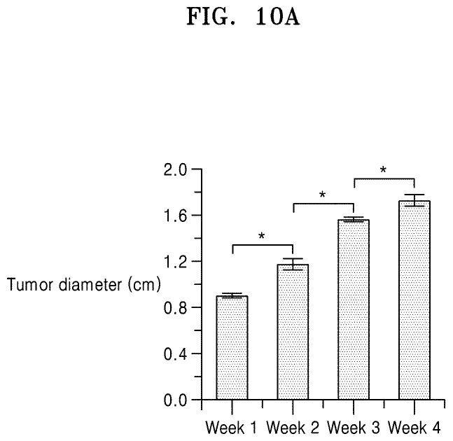

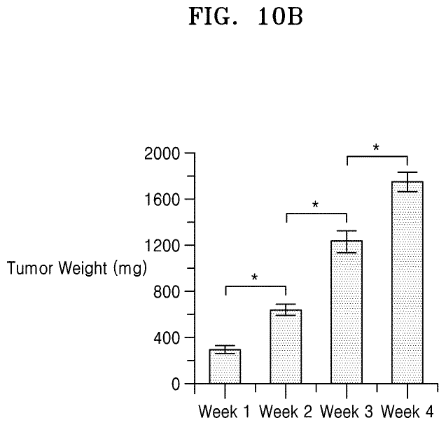

Then, the cultured 4T1 cancer cells were injected into the mammary fat pad of 8-week-old female BALB/C mice at a concentration of about 3×10⁶ cells/0.1 mL 1×PBS (pH 7.4). After the injection, the cells were subdivided into 4 groups (week-1, week-2, week-3, and week-4) of intervals of about one week according to the cancer progression period (4 weeks in total). In addition, a healthy mouse model that was not injected with anything was subdivided into 4 groups like the tumor models, and used as a control group. The sham control group, in which about 0.1 mL of 1×PBS (pH 7.4) was injected into the mammary fat pad of the mouse, was also subdivided into 4 groups like the tumor models and used. In addition, in order to confirm that the tumor models were well prepared, the tumor size and weight of the mouse tumor models were measured at intervals of about 1 week until 4 weeks after injection of 4T1 cancer cells, and the results are shown in FIG. 10. Specifically, the tumor diameter (cm) was measured after surgically isolating the tumor from the mouse tumor models (the diameter was measured at least twice in the direction perpendicular to each other over the largest part of the tumor),and after measuring the tumor diameter, the weight (mg) was measured by using an electronic scale. The average diameter of the tumor was calculated by using the following formula:

Formula=√(d1×d2) (d1 and d2 are the longest diameters of the tumor, which are diameters perpendicular to each other.)

As shown in FIG. 10, it was confirmed that both tumor size and weight were significantly increased from week 1 to week 4 after injection of 4T1 cancer cells. Therefore, it was confirmed that cancer was successfully formed in the mouse tumor model.

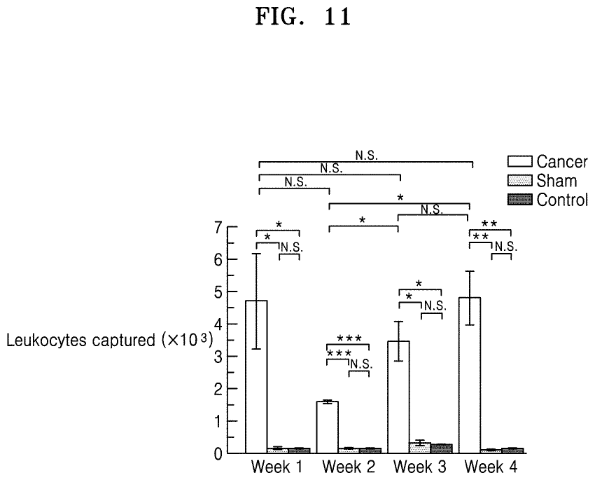

In the same manner as in Example 1, a microfluidic chip including a microfluidic channel in which mouse ICAM-1, mouse P-selectin, and mouse E-selectin, etc. were immobilized (each using a solution at a concentration of about 5 μg/ml) was prepared. After isolating leukocytes from the mice and washing the isolated leukocytes with 1×PBS, some of them were isolated separately, stained with Hoechst, and counted by using a hemocytometer and a fluorescence microscope, and the remaining leukocytes were diluted with 1×PBS to a final concentration (about 10⁶ cells/ml), and a biological sample including the leukocytes was prepared. The biological sample was injected into the microfluidic channel at a flow rate of about 8 μL/min for about 10 minutes, in order that about 80,000 leukocytes were injected into each channel. After the biological sample including the leukocytes was injected into the channel and flowed, the channel was washed for about 4 minutes by injecting 1×PBS into the channel at a flow rate of about 8 μl/min to remove the leukocytes that were not attached to the channel, and this was repeated about two more times. Then, in order to stain the leukocytes attached or trapped in the microfluidic channel, the inside of the channel was filled with dyes such as Hoechst and Cell tracker, and incubated at room temperature for about 20 minutes, and the channel was washed for about 4 minutes by injecting 1×PBS into the channel at a flow rate of about 8 μl/min. After that, the inside of each channel was photographed with a fluorescence microscope, and images inside each channel to which leukocytes were attached were captured, and the total number of leukocytes captured in the channel was counted, and the results are shown in FIG. 11.

As shown in FIG. 11, it may be seen that when leukocytes of cancer-induced mice were injected into the channel, significantly more leukocytes were attached to the channel and detected than when leukocytes of a control mouse or a sham control mouse were injected into the channel. Specifically, it was confirmed that the number of leukocytes attached to the channel was significantly increased in the case of all groups of mouse tumor models from week 1 to week 4 after injection of 4T1 cancer cells, compared to the mouse models in which tumor was not induced. In particular, it was confirmed that the tumor size gradually increased over the course of 4 weeks after injection of 4T1 cancer cells into mice (see FIG. 10), and the amount of leukocytes attached to the channel and detected also increased, among the leukocytes isolated from cancer-induced mice, as the tumor size of the cancer-induced mice increased.

These results indicate that in leukocytes in a disease state or in leukocytes in an abnormal state, expression of factors that interact with leukocyte extravasation factors increases or a number of leukocytes with increased expression of factors that interact with leukocyte extravasation factors increases, and it is possible to effectively diagnose a disease that increases expression of surface-related factors in a leukocyte, such as cancer, by using leukocyte extravasation factors, according to an embodiment. In addition, these results indicate that by using the leukocyte extravasation factors according to an embodiment, it is possible to quantitatively evaluate the progress of a disease, such as cancer, etc., in a subject with a disease that increase the expression of the surface-related factors of leukocytes, such as cancer, etc., or to make diagnosis on progression of a disease, such as cancer.

### EXPERIMENTAL EXAMPLE 7

### Confirmation of Proportion of Neutrophils Among the Detected Leukocytes

The proportion of neutrophils among the detected leukocytes was analyzed by using the leukocyte extravasation factors according to an embodiment.

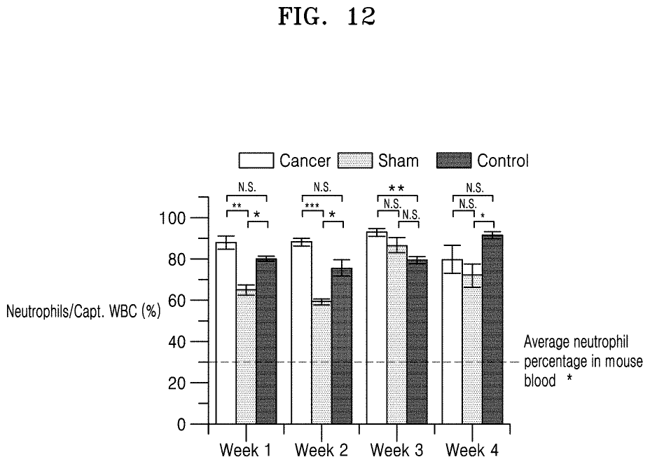

Specifically, leukocytes isolated from the mouse tumor model of Experimental Example 6 (experimental group) and the mouse models in which tumor was not induced (control group, sham control group) were injected into the microfluidic channel prepared as in Experimental Example 6, and then the leukocytes captured in each channel were analyzed as a target. More specifically, each microfluidic channel in which the leukocytes were captured was reacted with about 4% paraformaldehyde solution at room temperature for about 10 minutes to fix the captured leukocytes in the channel. After fixation, the channel was washed with 1×PBS, and 0.5% Triton-X solution was injected into the channel for about 10 minutes to increase reagent permeability, and then the channel was washed with 1×PBS. Afterwards, about 3% BSA was injected and reacted at room temperature for about 1 hour to block non-specific binding. Next, antibodies to which FITC fluorescence was attached targeting neutrophil myeloperoxidase were injected into the channel to identify neutrophils. In addition, in order to visualize all the leukocytes captured in the channel, the cells in the channel were stained with Hoechst reagent at about 4° C. for about 24 hours. After washing the channels with 1×PBS, the total number of leukocytes and the number of neutrophils captured in each channel were counted by using a fluorescence microscope, and the proportion of neutrophils in the captured leukocytes was calculated, and the results are shown in FIG. 12.

As shown in FIG. 12, it was confirmed that neutrophils were present in the highest proportion among the leukocytes captured in the microfluidic channel coated with leukocyte extravasation factors in all the mouse models of the experimental group, the control group, and the sham control group.

These results may mean that the majority of leukocytes interacting with the leukocyte extravasation factors according to an embodiment are neutrophils.

### EXPERIMENTAL EXAMPLE 8

### Detection and Analysis of Leukocytes with Bright Field

If captured leukocyte may be counted with a Bright field (BF) as well as with fluorescence images, analysis may be performed with a regular camera, etc., and the present disclosure may be more useful at the point-of-care, and thus, in this experimental example, detection and analysis of leukocytes by using a BF was performed.

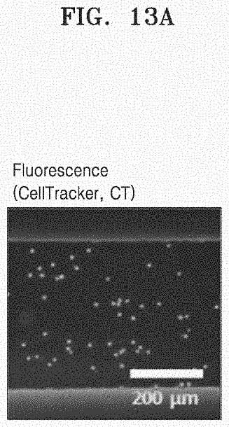

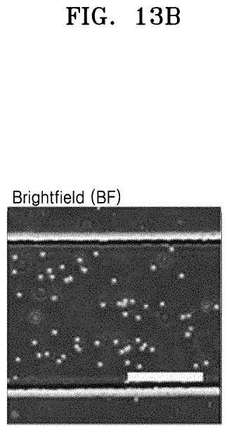

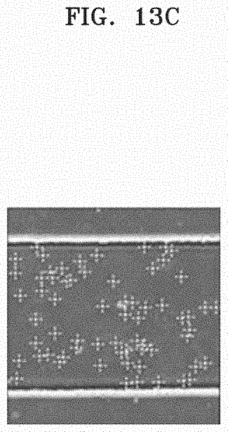

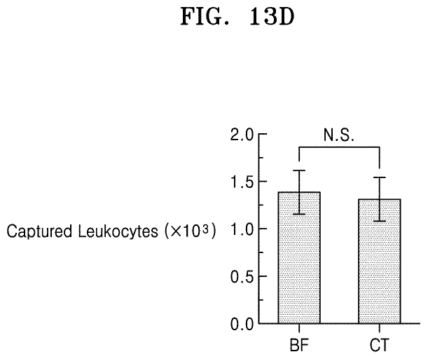

Specifically, in the same manner as in Experimental Example 1, blood of rats was collected about 12 hours after bacterial inoculation to isolate leukocytes, and the isolated leukocytes were injected into a microfluidic chip prepared in the same manner as in Example 1, including a microfluidic channel (each using a solution having a concentration of about 5 μg/ml), in which rat ICAM-1, rat P-selectin, and rat E-selectin were immobilized. Thereafter, in the same manner as in Experimental Example 1, after staining the cells captured in each channel using CellTracker, the cells were imaged with a fluorescence microscope, and imaged under a microscope in a BF mode, respectively, and (number of leukocytes attached to the channel)/(total number of leukocytes in the sample) % was measured in each image above, and the results are shown in FIG. 13.

As shown in FIG. 13, it was found that there was no statistical difference between the results of an analysis through the fluorescence image and an analysis with a BF, and it was found that a number of cells may be counted by imaging on the BF as by using fluorescence images. These results indicate that the method and device according to an embodiment allow on-site diagnosis of diseases or immune conditions without expensive and difficult-to-carry equipments.

Thus far, specific parts of the present disclosure are described in detail, and it will be apparent for those of ordinary skill in the art, that this specific description is only for preferred embodiments, and the scope of the present invention is not limited thereto. Accordingly, the substantial scope of the present disclosure will be defined by the appended claims and their equivalents.

1. A method of detecting leukocytes in a disease state, comprising:
contacting an isolated biological sample including leukocytes, or leukocytes isolated from the biological sample with leukocyte extravasation factors to capture leukocytes in a disease state in the sample by the leukocyte extravasation factors; and detecting the captured leucocytes.

2. The method of claim 1, wherein the leukocyte extravasation factors are immobilized on a wall of a channel, a surface of a particle, at least a portion of a vessel, or at least a portion of a well.

3. The method of claim 1 or 2, comprising counting a total number of leukocytes per unit sample volume in an isolated biological sample including the leukocytes, or isolating the leukocytes from the isolated biological sample including the leukocytes and counting the isolated leukocytes.

4. The method of claim 3, wherein the detecting comprises analyzing a ratio of the number of leukocytes captured by the leukocyte extravasation factors to the total number of counted leukocytes per unit sample volume, or analyzing the number of leukocytes captured by the leukocyte extravasation factors among the number of the isolated leukocytes.

5. The method of claim 1, wherein the leukocyte extravasation factors are at least one factor selected from the group consisting of selectins, CD34, intercellular adhesion molecule-1 (ICAM-1), soluble ICAM-1, ICAM-2, soluble ICAM-2, glycosylation-dependent cell adhesion molecule-1 (GlyCAM-1), mucosal vascular addressin cell adhesion molecule 1 (MadCAM-1), platelet/endothelial-cell-adhesion molecule (PECAM-1), junctional adhesion molecule A (JAM-A), JAM-B, JAM-C, endothelial cell-selective adhesion molecule (ESAM), vascular cell-adhesion molecule 1 (VCAM-1), cluster of differentiation 99 (CD99), integrins, extracellular domains thereof, cells including the same, membranes of cells including the same, and combinations thereof.

6. The method of claim 5, wherein the selectins are P-selectin, E-selectin, or a combination thereof.

7. The method of claim 2, wherein the leukocyte extravasation factors are immobilized to the channel, particle, vessel, or well by an immobilizing compound or a linker.

8. The method of claim 1, wherein the leukocytes in a disease state or a cell population of the leukocytes in a disease state have increased or decreased binding capacity with leukocyte extravasation factors, compared to leukocytes in a normal state or a cell population of the leukocytes in a normal state.

9. The method of claim 8, wherein the leukocytes in a disease state have increased or decreased expression or activity of factors capable of binding to leukocyte extravasation factors, compared to leukocytes in a normal state, or the cell population of the leukocytes in a disease state have increased or decreased expression or activity of factors capable of binding to leukocyte extravasation factors, compared to a cell population of the leukocytes in a normal state.

10. The method of claim 9, wherein the factors capable of binding to the leukocyte extravasation factors are at least one selected from the group consisting of sialylated carbohydrates, L-selectin, P-selectin glycoprotein ligand 1 (PSGL-1), and leukocyte function-associated antigen 1 (LFA-1), macrophage-1 antigen (Mac-1; integrin alpha M), VLA-4, CD24, CD44, and E-selectin ligand 1 (ESL-1).

11. The method of claim 1, wherein the disease state is inflammation, an infectious disease, an immune disease, cancer, or cancer metastasis.

12. The method of claim 11, wherein the infectious disease is systemic or local infections of viruses, bacteria, mold, or fungi, or sepsis, bacteremia, or viremia.

13. The method of claim 1, wherein the detecting is detecting by imaging the captured leukocytes, detecting by fluorescence staining the captured leukocytes, measuring isolated leukocyte lysates by lysing the captured leukocytes, or detecting by attaching a detectable label to the leukocytes or the leukocyte extravasation factors.

14. A method of providing information on diagnosis of disease related to leukocyte in the disease state, comprising: contacting an isolated biological sample including leukocytes, or leukocytes isolated from the biological sample with leukocyte extravasation factors to capture leukocytes in a disease state in the sample by the leukocyte extravasation factors; and detecting the captured leukocytes.

15. The method of claim 14, wherein the leukocyte extravasation factors are immobilized on a wall of a channel, a surface of a particle, at least a portion of a vessel, or at least a portion of a well.

16. The method of claim 14, wherein the leukocyte extravasation factors are at least one factor selected from the group consisting of selectins, CD34, intercellular adhesion molecule-1 (ICAM-1), soluble ICAM-1, ICAM-2, soluble ICAM-2, glycosylation-dependent cell adhesion molecule-1 (GlyCAM-1), mucosal vascular addressin cell adhesion molecule 1 (MadCAM-1), platelet/endothelial-cell-adhesion molecule (PECAM-1), junctional adhesion molecule A (JAM-A), JAM-B, JAM-C, endothelial cell-selective adhesion molecule (ESAM), vascular cell-adhesion molecule 1 (VCAM-1), cluster of differentiation 99 (CD99), integrins, extracellular domains thereof, cells the same, membranes of cells including the same, and combinations thereof.

17. The method of claim 16, wherein the selectins are P-selectin, E-selectin, or a combination thereof.

18. The method of claim 14, wherein the disease related to leukocyte is inflammation, an infectious disease, an immune disease, cancer, or cancer metastasis.

19. The method of claim 18, wherein the infectious disease is systemic or local infections of viruses, bacteria, mold, or fungi, or sepsis, bacteremia, or viremia.

20. A device for detecting leukocytes in a disease state, comprising a detector for detecting leukocytes in a disease state, comprising a channel, a particle, a vessel, or a well, in which leukocyte extravasation factors are immobilized on a wall of the channel, a surface of the particle, at least a portion of the vessel, or at least a portion of the well,
wherein leukocytes in a disease state in an isolated biological sample are captured by the leukocyte extravasation factors and detected.

21. The device of claim 20, wherein the leukocyte extravasation factors are at least one factor selected from the group consisting of selectins, CD34, intercellular adhesion molecule-1 (ICAM-1), soluble ICAM-1, ICAM-2, soluble ICAM-2, glycosylation-dependent cell adhesion molecule-1 (GlyCAM-1), mucosal vascular addressin cell adhesion molecule 1 (MadCAM-1), platelet/endothelial-cell-adhesion molecule (PECAM-1), junctional adhesion molecule A (JAM-A), JAM-B, JAM-C, endothelial cell-selective adhesion molecule (ESAM), vascular cell-adhesion molecule 1 (VCAM-1), cluster of differentiation 99 (CD99), integrins, cells including at least one thereof and membranes of the cells, extracellular domains thereof, and combinations thereof.

22. The device of claim 21, wherein the selectins are P-selectin, E-selectin, or a combination thereof.

23. The device of claim 20, wherein the leukocyte extravasation factors are immobilized to the channel, particle, vessel, or well by an immobilizing compound or a linker.

24. The device of claim 20, wherein the leukocytes in a disease state or a cell population of the leukocytes in a disease state have increased or decreased binding capacity with leukocyte extravasation factors, compared to leukocytes in a normal state or a cell population of the leukocytes in a normal state.

25. The device of claim 24, wherein the leukocytes in a disease state have increased or decreased expression or activity of factors capable of binding to leukocyte extravasation factors, compared to leukocytes in a normal state, or the cell population of the leukocytes in a disease state have increased or decreased expression or activity of factors capable of binding to leukocyte extravasation factors, compared to a cell population of leukocytes in a normal state.

26. The device of claim 25, wherein the factors capable of binding to the leukocyte extravasation factors are at least one selected from the group consisting of sialylated carbohydrates, L-selectin, P-selectin glycoprotein ligand 1 (PSGL-1), and leukocyte function-associated antigen 1 (LFA-1), macrophage-1 antigen (Mac-1; integrin alpha M), VLA-4, CD24, CD44, and E-selectin ligand 1 (ESL-1).

27. The device of claim 20, wherein the disease state is inflammation, an infectious disease, an immune disease, cancer, or cancer metastasis.

28. The device of claim 27, wherein the infectious disease is systemic or local infections of viruses, bacteria, mold, or fungi, or sepsis, bacteremia, or viremia.

29. The device of claim 20, wherein the detection is detecting by imaging the captured leukocytes, detecting by fluorescence staining the captured leukocytes, measuring isolated leukocyte lysates by lysing the captured leukocytes, or detecting by attaching a detectable label to the leukocytes or leukocyte extravasation factors.

30. A device for diagnosing a leukocyte-related disease, comprising a detector for detecting leukocytes in a disease state, comprising a channel, a particle, a vessel, or a well, in which leukocyte extravasation factors are immobilized on a wall of the channel, a surface of the particle, at least a portion of the vessel, or at least a portion of the well,
wherein the leukocytes in a disease state in an isolated biological sample are captured by the leukocyte extravasation factors and detected.

31. The device of claim 30, wherein the leukocyte extravasation factors are at least one factor selected from the group consisting of selectins, CD34, intercellular adhesion molecule-1 (ICAM-1), soluble ICAM-1, ICAM-2, soluble ICAM-2, glycosylation-dependent cell adhesion molecule-1 (GlyCAM-1), mucosal vascular addressin cell adhesion molecule 1 (MadCAM-1), platelet/endothelial-cell-adhesion molecule (PECAM-1), junctional adhesion molecule A (JAM-A), JAM-B, JAM-C, endothelial cell-selective adhesion molecule (ESAM), vascular cell-adhesion molecule 1 (VCAM-1), cluster of differentiation 99 (CD99), integrins, cells including at least one thereof and membranes of the cells, extracellular domains thereof, and combinations thereof.

32. The device of claim 31, wherein the selectins are P-selectin, E-selectin, or a combination thereof.

33. The device of claim 30, wherein the leukocyte-related disease is inflammation, an infectious disease, an immune disease, a metabolic disease, cancer, or cancer metastasis.

34. The device of claim 33, wherein the infectious disease is systemic or local infections of viruses, bacteria, mold, or fungi, or sepsis, bacteremia, or viremia.
